(19) United States
(12) Patent Application Publication
(10) Pub. No.: US 2003/0082150 A1
(43) Pub. Date: May 1, 2003

(54) RECOMBINANT ADENOVIRAL VECTORS FOR HUMAN TUMOUR GENE THERAPY

(76) Inventors: THIERRY BOON-FALLEUR, BRUXELLES (BE); MARIE-THERESE DUFFOUR, PARIS (FR); HEDI HADDADA, BOURG LA REINE (FR); CHRISTOPHE LURQUIN, BRUXELLES (BE); MICHEL PERRICAUDET, ECROSNES (FR); CATHERINE UYTTENHOVE-GHESQUIERE, CHAUMONT GISTOUX (BE); GUY WARNIER, LINKEBEEK (BE)

Correspondence Address: WILEY, REIN & FIELDING, LLP, INTELLECTUAL PROPERTY DEPARTMENT, 1776 K. STREET N.W., WASHINGTON, DC, 20006, US

(21) Appl. No.: 09/125,887

(22) PCT Filed: Mar. 12, 1997
(86) PCT No.: PCT/FR97/00435

(30) Foreign Application Priority Data
Mar. 14, 1996 (FR) 96/03207

(51) Int. Cl. A61K 48/00; C12N 15/861

(52) U.S. Cl. 424/93.21; 514/44; 435/320.1

(57) ABSTRACT

A method for treating human tumours by gene therapy is disclosed. In particular, defective recombinant viruses with a sequence coding for a human tumour-specific antigen, and the use thereof for treating or preventing human tumours, as well as producing specific cytotoxic T-cells (CTLs) in vitro or ex vivo, are disclosed. Pharmaceutical compositions comprising said viruses, particularly in injectable form, are also disclosed.

The present invention relates to a method for the treatment of human tumours by gene therapy. It relates especially to defective recombinant viruses carrying a sequence coding for an antigen specific to human tumours, and to their use for the preventive or curative treatment of human tumours and also for generating specific CTL in vitro or ex vivo. It also relates to pharmaceutical compositions containing these viruses, in particular in injectable form.

Gene therapy consists in correcting a deficiency or an abnormality by introducing genetic information into the affected cell or organ. This information may be introduced either in vitro into a cell extracted from the organ and then reinjected into the body, or in vivo, directly into the tissue in question. Being a negatively charged, high molecular weight molecule, DNA has difficulty in passing spontaneously through phospholipid cell membranes. Hence various vectors are used in order to permit gene transfer: viral vectors on the one hand, natural or synthetic chemical and/or biochemical vectors on the other hand. Chemical and/or biochemical vectors are, for example, cations (calcium phosphate, DEAE-dextran, etc.) which act by forming precipitates with DNA which can be “phagocytosed” by the cells. They can also be liposomes in which the DNA is incorporated and which fuse with the plasma membrane. Synthetic gene transfer vectors are generally cationic polymers or lipids which complex DNA and form with the latter a particle carrying positive surface charges. These particles are capable of interacting with the negative charges of the cell membrane, and then of crossing the latter. Dioctadecylamidoglycylspermine (DOGS, Transfectam™) or N-[1-(2,3-dioleyloxy)propyl]-N,N,N-trimethylammonium chloride (DOTMA, Lipofectin™) may be mentioned as examples of such vectors. Chimeric proteins have also been developed; they consist of a polycationic portion which condenses DNA, linked to a ligand which binds to a membrane receptor and draws the complex into the cells by endocytosis. It is thus theoretically possible to “target” a tissue or certain cell populations so as to improve the in vivo bioavailability of the transferred gene (for reviews, see Behr, 1993, Cotten and Wagner, 1993). Among the viruses which are potentially usable as vectors for gene transfer, retroviruses (RSV, HMS, MMS, and the like), the HSV virus, adeno-associated viruses and adenoviruses may be mentioned more especially. These viruses have all been used to infect different cell types.

Gene therapy approaches have been developed for the treatment of various types of pathology, including nervous system disorders, cardiovascular diseases or cancer. As regards the cancer field more especially, various approaches have been proposed in the prior art. Thus, some studies describe the use of lymphocytes activated ex vivo by culturing in the presence of interleukin-2 or by transfection with the interleukin-2 gene. Studies employing adoptive immuno-therapy have also been undertaken with monocytes-macrophages purified and activated ex vivo with interferon in order to increase their tumoricidal power and then reinjected into patients (Andressen et al., Cancer Res. 50 (1990) 7450). The possibility of using genetically modified macrophages has also been described (WO95/06120). Another series of approaches is based on the transfer of toxic genes capable of inducing the death of cancer cells directly or indirectly. This type of approach has been described, for example, with the thymidine kinase gene, transferred in vivo either by an adenoviral vector (PCT/FR94/01284; PCT/FR94/01285) or by grafting cells that produce a retroviral vector (Caruso et al., PNAS 90 (1993) 7024). Other genes used are, for example, the cytosine deaminase gene.

The present application relates to a new method for the treatment of cancer. It is intended most especially for the treatment of human tumours, and in particular melanomas. The method of the invention is based on the in vivo transfer and expression of antigens specific to human tumours such as melanomas, capable of inducing (i) an immune protection against the appearance of this type of cancer, and (ii) an expansion of the population of cytotoxic T cells (CTL) specific for cells possessing these antigens, and thus a destruction of the corresponding tumour cells by the immune system.

The immune system has, among other functions, the capacity to effect protection against viral infections. This capacity is discharged by cytotoxic T lymphocytes (CTL). CTL display two exceptional features: they are highly specific and of great efficacy. They destroy the infected cells after identifying a viral antigen at their surface. The antigen in question manifests itself in the form of a peptide combined with a major histocompatibility complex class I (MHC-I) molecule. In the context of tumours, if was observed, initially in mice, that these malignant cells possess peptide-MHC-I molecule complexes capable of producing, as in the context of antiviral responses, a CTL-mediated immune response. These peptides originate, in particular, from proteins encoded by genes which are mutated or activated selectively in the tumour cells. These proteins are designated tumour specific antigens. More recently, differentiation antigens recognized by CTL have been characterized on human tumours.

The present invention relates to a new method for the treatment of human tumours. It is the outcome, in particular, of the demonstration of vectors of viral origin capable of transferring and expressing in vivo antigens specific to human tumours or to melanomas. It is based more especially on the demonstration in mouse models that defective recombinant adenoviruses are capable of inducing an immunization against this type of antigen, enabling lymphocytic responses to these antigens, and in particular tumour cells carrying them, to be obtained in vivo. This method according to the invention hence makes it possible, by the transfer of these genes, to act on the development of human tumours in an especially effective manner, stopping their progression, it being possible to bring about eradication.

A first subject of the invention hence lies in a defective recombinant adenovirus containing, inserted into its genome, a nucleic acid coding for a tumour-specific protein or peptide, and more especially for all or part of an antigen specific to a melanoma.

Preferably, the antigen in question is specific to a human melanoma. Still more preferably, it is a fragment of an antigen specific to a human melanoma comprising the portion presented to the CTL in combination with MHC-I molecules. The antigens specific to human tumours have been described by Thierry Boon et al., (U.S. Pat. No. 5,342,774; U.S. Pat. No. 5,405,940; WO92/20356; WO94/23031; WO94/21126). These antigens, designated by the term MAGE, are expressed selectively in tumour cells, mainly human tumours. Various human MAGE genes have been described, and in particular the genes MAGE-1, MAGE-2, MAGE-3, MAGE-4, MAGE-5, MAGE-6, MAGE-7, MAGE-8, MAGE-9, MAGE-10, MAGE-11 and MAGE-12. As a representative example of homologous mouse genes, the S-MAGE-1 and S-MAGE-2 genes may also be quoted. As regards, more especially, BAGE, GAGE and RAGE genes, these are representative of other families of related genes.

According to a preferred embodiment, the present invention relates to a defective recombinant adenovirus containing, inserted into its genome, a nucleic acid coding for a protein, or peptide derived from the latter, selected from the proteins Mage-1, Mage-3, Bage, Gage and Rage. These antigens are, in effect, the most selective, in the sense that they are not detected, for the most part, on any non-tumoral somatic cell. The sequence of the antigen Mage-1 and of the corresponding gene have been described, in particular, in Van der Bruggen et al., Science 254 (1991) 1644. The sequence of the cDNA coding for Mage-1 and Mage-3 has been described, for example, in Gaugler et al., (J. Exp. Med. 179 (1994) 921).

As stated above, a preferred embodiment of the invention is represented by a defective recombinant adenovirus containing, inserted into its genome, a nucleic acid coding for a peptide of the protein Mage-1. Mage-3, Bage or Gage comprising the portion presented to the CTL in combination with MHC-I molecules. The Mage, Bage and Gage genes code, in effect, for large-sized proteins. These proteins are degraded by enzymatic digestion in the cell, leading to the generation of peptides. These peptides are the molecules which are then presented at the surface of the cells and which are recognized by the CTL in combination with MHC-I molecules (see FIG. 2). Still more preferably, the invention relates to a recombinant adenovirus comprising, inserted into its genome, a nucleic acid coding for a peptide of the protein Mage-1 or Mage-3 comprising the portion presented to the CTL.

According to a specific embodiment, the invention relates to a recombinant adenovirus comprising, inserted into its genome, the sequence SEQ ID No. 1. This sequence comprises the sequence coding for the nonapeptide (27 bp) of Mage-1 which is presented by the molecule HLA.A1 to the cytotoxic T lymphocytes. Still more preferably, the sequence in question is the sequence lying between residues 55 and 82 of the sequence SEQ ID No. 1.

According to another specific embodiment, the invention relates to a recombinant adenovirus comprising, inserted into its genome, the sequence SEQ ID No. 2. This sequence comprises the sequence coding for the nonapeptide (27 bp) of Mage-3 which is presented by the molecule HLA.A1 to the cytotoxic T lymphocytes.

According to another embodiment, the invention relates to [lacuna] recombinant adenovirus comprising, inserted into its genome, a nucleic acid coding for the antigenic peptide of the P1A gene of the DBA/2 mouse mastocytoma p815 (SEQ ID No. 3).

As stated above, the adenoviruses of the invention permit transfer and effective expression of these antigenic peptides in vivo. Thus, they make it possible, in a quite exceptional manner, to stimulate in vivo the appearance of cytotoxic T lymphocytes specific for these antigens, which selectively destroy any cell presenting this antigen at its surface.

Hence the viruses of the invention are usable for the preparation of pharmaceutical compositions intended for the treatment of cancers whose cells present Mage antigens at their surface. To prepare such compositions, a patient's tumour cells (generally from a melanoma) are preferably removed and analyzed in order (i) to determine the expression of a Mage gene for example, by RT-PCR, and (ii) where appropriate, to type this Mage antigen. An adenovirus containing a nucleic acid coding for all or part of the corresponding antigen is constructed and used for administration.

The viruses of the invention may also be used in vitro (or ex vivo) to generate populations of cytotoxic T cells specific for a given tumour antigen. To this end, a cell population is infected with a virus of the invention and then brought into contact with CTL cell precursors. The CTL cells specific for the antigens may then be selected in vitro, amplified and thereafter used as a medicinal product in order to destroy the corresponding tumours specifically. Advantageously, the cell population infected with a virus of the invention comprises antigen presenting cells (APC). These may be in particular macrophages (WO95/06120) or B cells.

In the adenoviruses of the invention, the inserted nucleic acid may be a fragment of complementary DNA (cDNA) or of genomic DNA (gDNA), or a hybrid construction consisting, for example, of a cDNA into which one or more introns might be inserted. It can also comprise synthetic or semi-synthetic sequences. As stated above, the nucleic acid in question codes for a whole protein, or peptide derived from this protein, selected from Mage-1, Mage-3, Bage and Gage. For the purposes of the present invention, the expression peptide derived from this protein means that the nucleic acid can code for just a fragment of the protein, it being necessary for this fragment to be capable of generating CTL. The fragment according to the invention hence carries at least one antigenic determinant recognized by a specific CTL. These fragments may be obtained by any technique known to a person skilled in the art, and in particular by genetic and/or chemical and/or enzymatic modifications, or alternatively by cloning by expression, permitting the selection of variants in accordance with their biological activity. Genetic modifications include suppressions, deletions, mutations, and the like.

The inserted nucleic acid is preferably a cDNA or from a gDNA.

Generally, the inserted nucleic acid also comprises sequences permitting the expression of the antigen or antigen fragment in the infected cell. The sequences can be ones which are naturally responsible for the expression of the said antigen when these sequences are capable of functioning in the infected cell. They can also be sequences of different origin, designated heterologous sequences (responsible for the expression of other proteins, or even synthetic sequences). In particular, the sequences can be promoters of eukaryotic or viral genes or derived sequences, stimulating or repressing the transcription of a gene specifically or non-specifically and inducibly or non-inducibly. As an example, they can be promoter sequences originating from the genome of the cell which it is desired to infect, or from the genome of a virus, and in particular the promoters of the adenovirus E1A and MLP genes, the CMV, RSV LTR, SRα promoter, and the like. Among eukaryotic promoters, there may also be mentioned the ubiquitous promoters (HPRT, vimentin, α-actin, tubulin, and the like), the promoters of intermediate filaments (desmin, neurofilaments, keratin, GFAP, and the like), the promoters of therapeutic genes (MDR, CFTR, factor VIII type, and the like), tissue-specific promoters (pyruvate kinase, villin, intestinal fatty acid binding protein promoter, smooth muscle cell α-actin promoter, promoters specific for the liver; Apo AI, Apo AII, human albumin, and the like) or alternatively promoters responding to a stimulus (steroid hormone receptor, retinoic acid receptor, and the like). In addition, these expression sequences may be modified by the addition of activation, regulatory, and the like, sequences. Moreover, when the inserted nucleic acid does not contain expression sequences, it may be inserted into the genome of the defective virus downstream of such a sequence.

The viruses according to the present invention are defective, that is to say incapable of replicating autonomously in the target cell. Generally, the genome of the defective viruses used in the context of the present invention hence lacks at least the sequences needed for replication of the said virus in the infected cell. These regions may be either removed (wholly or partially), or rendered non-functional, or replaced by other sequences, and in particular by the inserted gene. Preferably, the defective virus nevertheless retains the sequences of its genome which are needed for encapsidation of the viral particles.

The viruses according to the invention may be obtained from different serotypes of adenovirus. Different serotypes of adenovirus exist, the structure and properties of which vary somewhat. Among these serotypes, it is preferable to use, in the context of the present invention, human adenoviruses type 2 or 5 (Ad2 or Ad5) or adenoviruses of animal origin (see Application WO94/26914). Among adenoviruses of animal origin which are usable in the context of the present invention, adenoviruses of canine, bovine, murine (for example Mav1, Beard et al., Virology 75 (1990) 81), ovine, porcine, avian or alternatively simian (for example SAV) origin may be mentioned. Preferably, the adenovirus of animal origin is a canine adenovirus, more preferably a CAV2 adenovirus [Manhattan or A26/61 (ATCC VR-800) strain, for example]. It is preferable to use adenoviruses of human or canine or mixed origin in the context of the invention.

Preferably, the defective adenoviruses of the invention comprise the ITRs, a sequence permitting encapsidation and the nucleic acid of interest. Still more preferably, in the genome of the adenoviruses of the invention, the E1 region at least is non-functional. The viral gene in question may be rendered non-functional by any technique known to a person skilled in the art, and in particular by total elimination, substitution, partial deletion or addition of one or more bases in the gene or genes in question. Such modifications may be obtained in vitro (on the isolated DNA) or in situ, for example by means of genetic engineering techniques or alternatively by treatment by means of mutagenic agents. Other regions may also be modified, and in particular the E3 (WO95/02697), E2 (WO94/28938), E4 (WO94/28152, WO94/12649, WO95/02697) and L5 (WO95/02697) regions. According to a preferred embodiment, the adenovirus according to the invention comprises a deletion in the E1 and E4 regions. According to another preferred embodiment, it comprises a deletion in the E1 region, into which the E4 region and the nucleic acid are inserted (see FR94/13355). Advantageously, the deletion in the E1 region covers nucleotides 454 to 3328 (PvuII-BglII fragment) or 382 to 3446 (HinfII-Sau3A fragment). Advantageously, the deletion in the E4 region comprises at least the frames ORF3 and ORF6.

The nucleic acid of interest may be inserted at different regions of the adenovirus genome. The genome of an adenovirus is composed of a linear double-stranded DNA approximately 36 kb in size. It comprises, in particular, an inverted repeat sequence (ITR) at each end, an encapsidation sequence (Psi), early genes and late genes (see FIG. 1). The main early genes are contained in the E1, E2, E3 and E4 regions. Among these genes, those contained in the E1 region are needed for viral propagation. The main late genes are contained in the L1 to L5 regions. The genome of the Ad5 adenovirus has been completely sequenced and is accessible on a database (see, in particular, Genebank M73260). Similarly, portions or even the whole of other adenoviral genomes (Ad2, Ad7, Ad12, and the like) have also been sequenced. The nucleic acid of interest is preferably inserted into a region which is not essential to the production of the defective recombinant viruses. Thus, it is preferably inserted into the E1 region, which is defective in the virus and complemented by the producing line, into the E3 region, which is not essential to the production of the recombinant viruses (its inactivation does not need to be transcomplemented), or alternatively into the E4 region. In the latter case, it is necessary to complement the E4 functions during production, either by cotransfection with a helper virus or plasmid, or by means of a suitable line. Clearly, other sites may be used. In particular, access to the nucleotide sequence of the genome enables a person skilled in the art to identify regions enabling the nucleic acid of interest to be inserted.

The defective recombinant adenoviruses according to the invention may be prepared by any technique known to a person skilled in the art (Levrero et al., Gene 101 (1991) 195, EP 185 573; Graham, EMBO J. 3 (1984) 2917). Generally, the adenoviruses are produced by transfection of the DNA of the recombinant virus into a competent encapsidation cell line. The transfection may be a single one, when it is possible to have at one's disposal a construction carrying the whole of the genome of the recombinant virus, or, as is most often the case, a cotransfection of several DNA fragments supplying the different portions of the recombinant viral genome. In this case, the process involves one or more steps of homologous recombination between the different constructions in the encapsidation cell line, in order to generate the DNA of the recombinant virus. The different fragments used for the production of the virus may be prepared in different ways. The technique most generally used consists in isolating the viral DNA and then in modifying it in vitro by the standard methods of molecular biology (digestion, ligation, and the like). The constructions obtained are then purified and used to transfect the encapsidation lines. Another technique is based on the use of a plasmid carrying a portion of the genome of the recombinant virus, which is cotransfected with a virus supplying the missing portion of the genome. Another possibility lies in the use of prokaryotic plasmids to prepare the viral DNAs which are usable for the transfection (see Bett et al., PNAS 91 (1994) 8802, FR95/01632).

The cell line used should preferably (i) be transformable by the said elements, and (ii) contain the sequences capable of complementing the portion of the genome of the defective adenovirus, preferably in integrated form in order to avoid risks of recombination. As an example of a line, there may be mentioned the human embryonic kidney line 293 (Graham et al., J. Gen. Virol. 36 (1977) 59) which contains, in particular, integrated in its genome, the left-hand portion of the genome of an Ad5 adenovirus (12%) or lines capable of complementing the E1 and E4 functions, as are described, in particular, in Applications Nos. WO94/26914 and WO95/02697.

Thereafter, the adenoviruses which have multiplied are recovered and purified according to the standard techniques of molecular biology, as illustrated in the examples.

The present invention also relates to any pharmaceutical composition comprising one or more defective recombinant adenoviruses as described above. The pharmaceutical compositions of the invention may be formulated for the purpose of oral, parenteral, intranasal, intravenous, intramuscular, subcutaneous, transdermal, intratracheal, intraperitoneal, and the like, administration.

The present invention also relates to any pharmaceutical composition comprising cells infected with a defective recombinant adenovirus as described above. Advantageously, the composition of the invention comprises antigen presenting cells (APC) infected with a defective recombinant adenovirus as described above. As a specific example, there may be mentioned macrophages or B lymphocytes. The invention also relates to a composition comprising tumour antigen-specific cytotoxic T cells (CTL) prepared by culturing precursor cells in the presence of antigen presenting cells (APC) infected with a defective recombinant adenovirus as described above.

Preferably, a pharmaceutical composition of the invention contains vehicles which are pharmaceutically acceptable for an injectable formulation. These can be, in particular, sterile, isotonic saline solutions (containing monosodium or disodium phosphate, sodium, potassium, calcium or magnesium chloride and the like, or mixtures of such salts), or dry, in particular lyophilized, compositions which, on adding sterilized water or physiological saline, as the case may be, enable injectable solutions to be made up.

The doses of virus used for injection may be adapted in accordance with different parameters, and in particular in accordance with the mode of administration used, the pathology in question, the gene to be expressed or alternatively the desired period of treatment. Generally speaking, the recombinant adenoviruses according to the invention are formulated and administered in the form of doses of between 10⁴ and 10¹⁴ pfu, and preferably of 10⁶ to 10¹⁰ pfu. The term pfu (plaque forming unit) corresponds to the infectious power of a solution of virus, and is determined by infecting a suitable cell culture and measuring, generally after 15 days, the number of plaques of infected cells. The techniques of determination of the pfu titre of a viral solution are well documented in the literature.

Depending on the antigen in question, the adenoviruses of the invention may be used for the treatment or prevention of cancer, including, in particular, human tumours (for the antigens Mage-l to Mage-12, Gage and Bage and Rage) and sarcomas (for the Mage-1 antigens).

The present invention will be described more completely by means of the examples which follow, which are to be regarded as illustrative and non-limiting.

### LEGEND TO THE FIGURES

FIG. 1: Genetic organization of the AdS adenovirus.

FIG. 2: Expression and processing of the Mage antigens.

FIG. 3: Construction of the plasmids pAd.SRα-MAGE.

FIG. 4: Protocol No. 1 for immunization of DBA/2 mice with an Ad-P1A or control.

FIG. 5: Protocol No. 2 for immunization of DBA/2 mice with an Ad-P1A or control.

Table 1: Demonstration of the specific lysis by CTL of cells infected with an Ad-Mage according to the invention.

Table 2: Demonstration of the capacity of cells infected with an Ad-Mage according to the invention to stimulate the production of TNF by a CTL clone.

Table 3: Demonstration of the immunization of DBA/2 mice by injection, according to Protocol 1, of an Ad-P1A (Table 3A) or of a control adenovirus, Ad-βGal (Table 3B).

Table 4: Demonstration of the immunization of DBA/2mice by injection, according to Protocol 2, of an Ad-P1A (Table 4A) or of a control adenovirus, Ad-βGal (Table 4B)

General Techniques of Cloning and of Molecular Biology

The traditional methods of molecular biology, such as centrifugation of plasmid DNA in a caesium chloride/ethidium bromide gradient, digestion with restriction enzymes, gel electrophoresis, transformation in E.coli, precipitation of nucleic acids and the like, are described in the literature (Maniatis et al., 1989).

Enzymes were supplied by New England Biolabs (Beverly, Mass.).

To carry out ligation, the DNA fragments are separated according to their size on 0.8 to 1.5% agarose gels, purified with GeneClean (BI0101, La Jolla Calif.) and incubated overnight at 14° C. in a buffer comprising 50 mM Tris-HCl pH 7.4, 10 mM MgCl₂, 10 mM DTT, 2 mM ATP, in the presence of phage T4 DNA ligase.

Amplification by PCR (polymerase chain reaction) was also carried out according to Maniatis et al., 1989, with the following specifications:

MgCl₂ concentration brought to 8 mM;

Denaturation temperature 95° C., hybridization temperature 55° C., elongation temperature 72° C. This cycle was repeated 25 times in a PE9600 Thermalcycler (Perkin Elmer, Norwalk Colo.).

Oligonucleotides are synthesized using phosphoramidite chemistry in which the phosphoramidites are protected at the β position with a cyanoethyl group (Sinha et al., 1984, Giles 1985), with an Applied Biosystem model 394 automatic DNA synthesizer (Applied Biosystem, Foster City Calif.), according to the manufacturer's recommendations.

Sequencing was performed on double-stranded templates by the chain termination method using fluorescent primers. We used the Taq Dye Primer Kit sequencing kit from Applied Biosystem (Applied Biosystem, Foster City Calif.) according to the manufacturer's specifications.

### Example 1

### Construction of a Defective Recombinant Adenovirus Coding for a P1A Antigen Fragment

This example describes the construction of a defective recombinant adenovirus according to the invention coding for a fragment of the antigen P1A. More especially, the adenovirus carries the sequence SEQ ID No. 3. The adenovirus constructed is an adenovirus of serotype 5, possessing a deletion in the E1 and E3 regions, the nucleic acid of interest being inserted into the E1 region, at the level of the deletion.

The nucleic acid inserted into the E1 region comprises more especially:

the SRα promoter. The SRα promoter comprises the early origin of replication of SV40 and a portion of the HTLV1 LTR (corresponding to the domain R and to a portion of U5), followed by the 16S splice junction of SV40 (Takebe et al., Mol. Cell. Biol. 8 (1988) 466).

a P1A minigene of 44 bp (SEQ ID No. 3).

the polyadenylation site of the SV40 virus.

This nucleic acid was extracted from the plasmid pcD-SRα-P1A, corresponding to the plasmid pcD-SRα into which the P1A minigene has been cloned at the EcoRI site. The insert obtained was then cloned into the plasmid pAd.RSV-βGal (Stratford-Perricaudet et al., J. Clin. Invest. 90 (1992) 626), in place of the fragment containing the RSV LTR and the LacZ gene.

The plasmid pAd-SRα-P1A thereby obtained was then used to produce the recombinant adenovirus. To do this, line 293 cells were cotransfected with 5 μg of plasmid pAd-SRα-P1A and with 5 μg of the DNA of the mutant adenovirus dl 324 in the presence of calcium phosphate. The recombinant adenoviruses produced were then selected by plaque purification. After isolation, the recombinant adenovirus is amplified in the cell line 293, leading to a culture supernatant containing the unpurified recombinant adenovirus having a titre of approximately 10¹⁰ pfu/ml.

The viral particles are then purified by centrifugation on a caesium chloride gradient according to known techniques (see, in particular, Graham et al., Virology 52 (1973) 456). Analysis of the viral DNA by digestion using EcoRI restriction enzymes demonstrates the presence of the insert in the genome. The adenovirus may be stored at −80° C. in 10% glycerol.

### Example 2

### Construction of a Defective Recombinant Adenovirus Coding for a Mage-1 Antigen Fragment

This example describes the construction of a defective recombinant adenovirus according to the invention coding for a fragment of the antigen Mage-1. More especially, the adenovirus carries the sequence SEQ ID No. 1 coding for a fragment carrying the antigenic nonapeptide of Mage-1. The adenovirus constructed is an adenovirus serotype 5, possessing a deletion in the E1 and E3 regions, the nucleic acid of interest being inserted into the E1 region where the deletion is present.

The nucleic acid inserted into the E1 region comprises, more especially:

the SRα promoter. The SRα promoter comprises the early origin of replication of SV40 and a portion of the HTLV1 LTR (corresponding to the domain R and to a portion of U5), followed by the 16S splice junction of SV40 (Takebe et al., Mol Cell Biol 8 (1988) 466).

a MAGE-1 minigene of 116 bp (SEQ ID No. 1). This fragment was obtained by PCR from the complete Mage-1 gene. It contains an ATG at position 15, a stop codon at position 121 and a portion of exon 3 of the Mage-1 gene. It comprises the sequence corresponding to the nonapeptide (27 bp) which is presented by the HLA.A1 molecule to the cytotoxic T lymphocytes (see FIG. 2).

the polyadenylation site of the SV40 virus.

This nucleic acid was extracted from the plasmid pcD-SRα-MAGE-1, corresponding to the plasmid pcD-SRα into which the Mage-1 minigene has been cloned at the EcoRI site. The insert obtained was then cloned into the plasmid pAd.RSV-βGal (Stratford-Perricaudet et al., J. Clin. Invest. 90 (1992) 626), in place of the fragment containing the RSV LTR and the LacZ gene (FIG. 3).

The plasmid pAd-SRα-MAGE-1 thereby obtained was then used to produce the recombinant adenovirus. To this end, line 293 cells were cotransfected with plasmid pAd-SRα-MAGE-1 and with the DNA of the mutant adenovirus dl 324 in the presence of calcium phosphate. The recombinant adenoviruses produced were then selected by plaque purification. After isolation, the recombinant adenovirus is amplified in the cell line 293, leading to a culture supernatant containing the unpurified recombinant adenovirus having a titre of approximately 10¹⁰ pfu/ml.

The viral particles are then purified by centrifugation on a caesium chloride gradient according to known techniques (see, in particular, Graham et al., Virology 52 (1973) 456). Analysis of the viral DNA by digestion using EcoRI restriction enzymes demonstrates the presence of the insert in the genome. The adenovirus may be stored at −80° C. in 10% glycerol.

### Example 3

### Construction of a Defective Recombinant Adenovirus Coding for a Mage-3 Antigen Fragment

This example describes the construction of a defective recombinant adenovirus according to the invention coding for a fragment of the antigen Mage-3. More especially, the adenovirus carries the sequence SEQ ID No. 2 coding for a fragment carrying the antigenic nonapeptide of Mage-3. The adenovirus constructed is an adenovirus serotype 5, possessing a deletion in the E1 and E3 regions, the nucleic acid of interest being inserted into the E1 region where the deletion is present.

The nucleic acid inserted into the E1 region comprises, more especially:

the SRα promoter. The SRα promoter comprises the early origin of replication of SV40 and a portion of the HTLV1 LTR (corresponding to the domain R and to a portion of U5), followed by the 16S splice junction of SV40 (Takebe et al., Mol. Cell. Biol. 8 (1988) 466).

a MAGE-3 minigene of 44 bp (SEQ ID No. 2). This fragment was obtained by PCR from the complete Mage-3 gene. It contains an ATG at position 12, a stop codon at position 44 and the sequence corresponding to the nonapeptide (27 bp) which is presented by the HLA.A1 molecule to the cytotoxic T lymphocytes (see FIG. 2).

the polyadenylation site of the SV40 virus.

This nucleic acid was extracted from the plasmid pcD-SRα-MAGE-3, corresponding to the plasmid pcD-SRα into which the Mage-3 minigene has been cloned at the EcoRI site. The insert obtained was then cloned into the plasmid pAd.RSV-βGal in place of the fragment containing the RSV LTR and the LacZ gene (FIG. 3).

The plasmid pAd-SRα-MAGE-3 thereby obtained was then used to produce the recombinant adenovirus. To this end, line 293 cells were cotransfected with plasmid pAd-SRα-MAGE-3 and with the DNA of the mutant adenovirus dl324 in the presence of calcium phosphate. The recombinant adenoviruses produced were then selected by plaque purification. After isolation, the recombinant adenovirus is amplified in the cell line 293, leading to a culture supernatant containing the unpurified recombinant adenovirus having a titre of approximately 10¹⁰ pfu/ml.

The viral particles are then purified by centrifugation on a caesium chloride gradient according to known techniques (see, in particular, Graham et al., Virology 52 (1973) 456). Analysis of the viral DNA by digestion using EcoRI restriction enzymes demonstrates the presence of the insert in the genome. The adenovirus may be stored at −80° C. in 20% glycerol.

### Example 4

### Functional Characterization of the Adenoviruses of the Invention

This example demonstrates that the viruses according to the invention are capable of inducing the expression of the gene of interest coding for a protein whose degradation leads to the expression of an antigenic peptide at the surface of the target cells.

The expression of the MAGE-1 minigene and the presentation of the peptide were demonstrated on cells infected with Ad-Mage (4.1.), by determination of specific lysis (4.2.) and stimulation of TNF production (4.3.).

4.1. Cell Lines

The cell lines which have been infected are the following:

C1R.A1: B lymphocyte line transformed with EBV (ref. Storkus, W. J., Howell, D. N., Salter, R. D., Dawson, J. R., and Cresswell, P.: NK susceptibility varies inversely with target cell class I HLA antigen expression. J. Immunol. 138: 1675-1659, 1987) and transfected with the HLA.A1 gene cloned into the plasmid pHEBO.

Gerl III β E⁻F⁻: HLA.A1 human melanoma cells immunoselected for the loss of the antigen MAGE-1 (designated Gerlach E⁻ in Tables 1 and 2).

Hence these two lines express the HLA.A1 molecule but not the antigen MAGE-1.

4.2. Determination of Specific Lysis

This example demonstrates the existence of a specific lysis of the cells by a CTL clone specific for the antigen (radioactive chromium release test). To this end, the cells mentioned in 4.1. were infected with the adenovirus Ad-Mage-1 (Example 2) or with a control adenovirus (Ad-βGal) at a multiplicity of infection of 500 pfu/cell. The infected cells were then labelled with chromium-51 and thereafter incubated for 4 hours, on the basis of 1000 cells/well, with the specific CTL (clone 82:30) at different effector cells/target cell (E/T) ratios. The percentage lysis was then determined. The results obtained are presented in Table 1. They show clearly that cells infected with the viruses according to the invention display a sensitivity to lysis by the specific CTL which is markedly greater than that of cells infected with the control adenovirus. These cells also display a markedly enhanced sensitivity relative to cells transfected directly with the antigen Mage-1, thereby demonstrating the therapeutic efficacy of the vectors of the invention.

Hence these results show clearly that the viruses of the invention are capable of endowing cells with a considerable sensitivity to lysis by the specific CTL.

4.3. Stimulation of TNF Production

In this example, the capacity of Gerl III β E⁻F⁻ cells to stimulate the production of TNF by the same CTL clone was evaluated. To this end, Gerl III β E⁻F⁻ cells were infected with the adenovirus Ad-Mage-1(Example 2) or with a control adenovirus (Ad-β-Gal) at a multiplicity of infection of 50 or 100 pfu/cell, and then incubated with the CTL clone. After 24 hours, the amount of TNF present in the supernatants was measured by determination of their cytotoxicity on a TNF-sensitive line (line WEHI-164-13). Cell viability was measured by means of a calorimetric test (MTT). The results are expressed as optical density and then as quantity of TNF (pg/ml). This test was not performed on C1R.A1 cells, since they secrete TNF naturally. The Gerlach E⁺ control cells are melanoma cells expressing Mage-1 and HLA-A1.

The results obtained are presented in Table 2. They show clearly that the cells infected with the viruses according to the invention induce a production of TNF by the CTL, thereby confirming unambiguously the biological and therapeutic properties of the viruses of the invention.

### Example 5

### In Vivo Activity of the P1A Viruses of the Invention

Example 4 showed that, in vitro or ex vivo, cells infected with an adenovirus according to the invention are indeed recognized in a TNF test, and lysed in a test of radioactive chromium release by a specific CTL.

This example now demonstrates that, in vivo, the adenoviruses according to the invention are capable of generating a specific CTL response. More especially, this example demonstrates that 2 injections one week apart of 10⁹ viral particles (pfu) into DBA/2 mice generate a strong specific CTL response in a portion of them.

Two series of experiments were carried out. The plans of these two series are presented in FIGS. 4 and 5. In the first series of experiments (see plan FIG. 4), half of the viral particles were administered into the peritoneal cavity and the other half under the skin (at 4 sites). In the second series of experiments (see plan FIG. 5), the viral particles were administered by subcutaneous, intraperitoneal, intranasal and intratracheal injections. These two series of experiments were carried out according to the following protocol. The mice were sacrificed 15 days after the second series of injections of the adenovirus. The spleen cells of these mice were then brought into contact with the antigen P815A by means of L1210A+ cells (syngeneic leukaemia transfected with the P₁A gene of the P815 mastocytoma) irradiated for a period of 8 days. On completion of this mixed lymphocyte-tumour culture (MLTC), lymphocytes directed against the antigen P815A have proliferated and have differentiated into killer T lymphocytes. The latter are then brought into contact with cells labelled with radioactive chromium. The cells in question are P511 cells, an azaguanine-resistant variant of the P815 mouse mastocytoma carrying the A antigen, and P1.204 cells, a variant of this same mastocytoma which has lost the A antigen. The latter serves as a negative control. In order to eliminate all possibility of non-specific reaction, the target cells labelled with radioactive chromium are also brought into contact with syngeneic cells (L1210) carrying at their surface the antigens of the stimulating cell in the MLTC, with the exception of the A antigen of P815.

The results obtained with the first series of experiments, expressed as a percentage of specific lyses, are presented in Tables 3A and 3B.

The results obtained with the second series of experiments, expressed as a percentage of specific lyses, are presented in Tables 4A and 4B.

In both cases, the results presented show, mouse by mouse, levels of lysis which are proportional to effectors/targets ratios (means of duplicates). CTLs were obtained whatever the site of injection of the adenovirus (Table 4A +B). These results show clearly that the adenoviruses of the invention are capable of generating in vivo an immunity against cells carrying the tumour antigen.

TABLE 1

| | C1R | C1R | C1R trans- | Gerlach E- | Gerlach E- |
|---|---|---|---|---|---|
| | infected | infected | fected with | infected | infected |
| E/T ratio | Ad Mage-1 | Ad β gal | Mage-1 | Ad Mage-1 | Ad β gal |
| 30 | 62 | 3 | 33 | 15 | 0 |
| 10 | 59 | 2 | 40 | 15 | 0 |
| 3 | 61 | 4 | 45 | 13 | 0 |
| 1 | 44 | 3 | 30 | 6 | 0 |
| 0.3 | 44 | 1 | 25 | 5 | 0 |
| 0.1 | 28 | 2 | 19 | 2 | 2 |
| spontaneous 100 μl super- | 143 | 173 | 209 | 860 | 1049 |
| maximum natant | 1563 | 1958 | 1618 | 8320 | 7334 |
| spont/max | 9% | 9% | 13% | 10% | 14% |
| % of blue cells | | 40% | | | 100% |
| pfu/c | 500 | 500 | | 500 | 500 |

TABLE 2

| | | P82:30 | |
|---|---|---|---|
| | | Anti-Mage-1 CTL clone | |
| Gerlach | pfu/cell | −CTL | +CTL |
| Gerlach E+ (HLA A1+) (Mage 1+) | 16 | 1.049 = 0 pg TNF | 0.17 = 45 pg TNF |
| Gerlach E− (HLA A1+) (Mage 1−) | 50 | 1.082 = 0 pg TNF | 0.995 = 0 pg TNF |
| infected with Ad. βgal | | | |
| Gerlach E− (HLA A1+) (Mage 1−) | 100 | 1.045 = 0 pg TNF | 0.98 = 0 pg TNF |
| infected with Ad. βgal | | | |
| Gerlach E− (HLA A1+) (Mage 1−) | 50 | 1.015 = 0 pg TNF | 0.58 = 4 pg TNF |
| infected with Ad. Mage 1 | | | |
| Gerlach E− (HLA A1+) (Mage 1−) | 100 | 1.028 = 0 pg TNF | 0.355 = 12 pg TNF |
| infected with Ad. Mage 1 | | | |
| Controls Medium | | 1.102 = 0 pg TNF | |
| CTL P62.30 | | | 1.086 = 0 pg TNF |

| | |
|---|---|
| % of blue | Gerlach: 40 to 50% with 50 pfu and 60 to 70% with |
| cells | 100 pfu |

TABLE 3A
1°: mice injected with the recombinant adenovirus P1A

| | | | | L1210A++ | L1210+ | | |
|---|---|---|---|---|---|---|---|
| | | | | cold | cold | | |
| | | | | L1210 | L1210 | | |
| | | | | (50 cold targets | | | |
| | effector | | P- | per labelled | | L- | L- |
| | /target | P511 | 1.204 | target) | | 1210A+ | 1210 |
| Mouse | 218 | 14 | 10 | 4 | 0 | 15 | 21 |
| No. 1 | 71 | 6 | 0 | 3 | 2 | 7 | 13 |
| | 24 | 5 | 0 | 0 | 0 | 6 | 1 |
| | 8 | 2 | 0 | 0 | 0 | 3 | 11 |
| | 3 | 4 | 2 | 0 | 0 | 0 | 5 |
| | 1 | 2 | 0 | 0 | 0 | 6 | 0 |
| Mouse | 325 | 77 | 44 | 100 | 30 | 96 | 74 |
| No. 2 | 108 | 83 | 36 | 78 | 0 | 96 | 65 |
| | 36 | 69 | 24 | 56 | 1 | 78 | 50 |
| | 12 | 59 | 14 | 29 | 0 | 47 | 35 |
| | 4 | 37 | 3 | 3 | 4 | 23 | 20 |
| | 1 | 14 | 0 | 0 | 0 | 8 | 19 |
| Mouse | 250 | 76 | 34 | 64 | 10 | 81 | 92 |
| No. 3 | 83 | 63 | 34 | 41 | 8 | 88 | 65 |
| | 28 | 42 | 23 | 13 | 5 | 59 | 54 |
| | 9 | 25 | 8 | 1 | 0 | 13 | 24 |
| | 3 | 14 | 1 | 0 | 0 | 6 | 1 |
| | 1 | 2 | 0 | 0 | 0 | 1 | 8 |
| Mouse | 550 | 66 | 45 | 77 | 25 | 85 | 66 |
| No. 4 | 183 | 55 | 40 | 42 | 20 | 74 | 41 |
| | 61 | 41 | 28 | 22 | 2 | 47 | 40 |
| | 20 | 25 | 7 | 7 | 1 | 27 | 43 |
| | 7 | 12 | 7 | 0 | 0 | 22 | 16 |
| | 2 | 6 | 2 | 1 | 0 | 5 | 6 |

| | |
|---|---|
| MTD/GW | 5A94 |

TABLE 3B
2°: mice injected with the recombinant adenovirus βgal

| | | | | L1210A++ | L1210+ | | |
|---|---|---|---|---|---|---|---|
| | | | | cold L1210 | cold L1210 | | |
| | effector/ | | | (50 cold targets per | | | |
| | target | P511 | P1.204 | labelled target) | | L1210A+ | L1210 |
| Mouse No. 1 | 375 | 1 | 2 | 2 | 0 | 11 | 20 |
| | 125 | 5 | 5 | 0 | 0 | 6 | 2 |
| | 42 | 3 | 0 | 1 | 0 | 1 | 6 |
| | 14 | 4 | 0 | 0 | 0 | 1 | 3 |
| | 5 | 2 | 1 | 0 | 0 | 0 | 1 |
| | 2 | 0 | 0 | 0 | 0 | 0 | 1 |
| Mouse No. 2 | 463 | 11 | 0 | 10 | 0 | 12 | 14 |
| | 154 | 6 | 0 | 0 | 0 | 10 | 0 |
| | 51 | 5 | 3 | 0 | 0 | 2 | 1 |
| | 17 | 2 | 0 | 0 | 0 | 3 | 7 |
| | 6 | 2 | 0 | 1 | 2 | 2 | 0 |
| | 2 | 3 | 0 | 0 | 0 | 0 | 5 |
| Mouse No.3 | 438 | 4 | 0 | 0 | 0 | 8 | 12 |
| | 146 | 7 | 2 | 0 | 0 | 1 | 11 |
| | 49 | 0 | 0 | 5 | 0 | 0 | 7 |
| | 16 | 2 | 0 | 0 | 0 | 3 | 4 |
| | 5 | 0 | 0 | 0 | 0 | 0 | 1 |
| | 2 | 2 | 0 | 0 | 0 | 0 | 0 |
| Mouse No. 4 | 488 | 6 | 2 | 2 | 0 | 13 | 4 |
| | 163 | 8 | 1 | 0 | 0 | 0 | 0 |
| | 54 | 6 | 0 | 0 | 0 | 2 | 11 |
| | 18 | 1 | 0 | 1 | 0 | 3 | 0 |
| | 6 | 4 | 0 | 0 | 0 | 2 | 4 |
| | 2 | 2 | 0 | 0 | 0 | 0 | 6 |
| | spontaneous | 236 | 194 | 140 | 116 | 125 | 95 |
| | maximum | 1722 | 1170 | 786 | 547 | 790 | 449 |
| | spont/max | 14% | 16% | 10% | 21% | 16% | 21% |

5A94

TABLE 4a

| | | | | P511+ | P1.204+ |
|---|---|---|---|---|---|
| | Effector/ | | | cold L1210 | cold L1210 |
| | target | P511 | P1.204 | 50><1 ratio | 50><1 ratio |
| Subcutaneous | | | | | |
| Mouse | 490 | 9 | 2 | 0 | 1 |
| No. 1 | 163 | 5 | 3 | 0 | 0 |
| | 54 | 3 | 1 | 0 | 0 |
| | 18 | 3 | 0 | 0 | 0 |
| | 8 | 2 | 1 | 0 | 0 |
| | 2 | 0 | 0 | 0 | 0 |
| | | | | 0 | |
| Mouse | 50 | 5 | 0 | 0 | 1 |
| No. 2 | 27 | 3 | 0 | 3 | 0 |
| | 9 | 5 | 0 | 0 | 0 |
| | 3 | 6 | 0 | 1 | 0 |
| | 1 | 4 | 0 | 0 | 0 |
| | 0.3 | 2 | 0 | | 0 |
| | | | | 68 | |
| Mouse | 300 | 73 | 18 | 72 | 0 |
| No. 3 | 100 | 71 | 15 | 75 | 0 |
| | 33 | 92 | 10 | 57 | 1 |
| | 11 | 73 | 8 | 25 | 8 |
| | 4 | 56 | 3 | 8 | 0 |
| | 1 | 25 | 2 | | 0 |
| Intra- | | | | | |
| peritoneal | | | | | |
| Mouse | 258 | 51 | 9 | 48 | 0 |
| No. 1 | 85 | 43 | 10 | 37 | 0 |
| | 28 | 43 | 8 | 27 | 0 |
| | 9 | 38 | 3 | 12 | 1 |
| | 3 | 18 | 2 | 8 | 0 |
| | 1 | 7 | 0 | 1 | 0 |
| Mouse | 295 | 9 | 4 | 9 | 2 |
| No. 2 | 88 | 8 | 6 | 2 | 0 |
| | 33 | 7 | 4 | 2 | 0 |
| | 11 | 3 | 2 | 1 | 0 |
| | 4 | 1 | 2 | 0 | 0 |
| | 1 | 0 | 0 | 0 | 1 |
| Mouse | 400 | 68 | 12 | 76 | 0 |
| No. 3 | 133 | 77 | 12 | 82 | 0 |
| | 44 | 88 | 14 | 73 | 0 |
| | 15 | 14 | 7 | 66 | 0 |
| | 6 | 10 | 6 | 30 | 0 |
| | 3 | 10 | 1 | 14 | 0 |
| Mouse | 185 | 8 | 2 | 3 | 0 |
| No. 4 | 52 | 3 | 0 | 3 | 0 |
| | 57 | 10 | 2 | 3 | 0 |
| | 8 | 4 | 1 | 2 | 0 |
| | 2 | 3 | 0 | 0 | 0 |
| | 0.8 | 2 | 0 | 2 | 0 |
| Intratracheal | | | | | |
| Mouse | 440 | 67 | 11 | 50 | 3 |
| No. 1 | 147 | 78 | 13 | 52 | 2 |
| | 49 | 64 | 6 | 49 | 0 |
| | 18 | 34 | 8 | 23 | 0 |
| | 6 | 31 | 7 | 9 | 0 |
| | 2 | 14 | 0 | 2 | 0 |
| Mouse | 410 | 8 | 7 | 3 | 2 |
| No. 2 | 137 | 6 | 7 | 3 | 1 |
| | 45 | 6 | 4 | 0 | 1 |
| | 15 | 5 | 2 | 2 | 0 |
| | 9 | 1 | 3 | 0 | 0 |
| | 2 | 1 | 2 | 0 | 2 |
| Mouse | 344 | 8 | 2 | 3 | 1 |
| No. 3 | 115 | 4 | 0 | 2 | 0 |
| | 38 | 8 | 0 | 1 | 0 |
| | 13 | 8 | 0 | 0 | 0 |
| | 4 | 4 | 1 | 0 | 0 |
| | 1 | 1 | 0 | 0 | 0 |
| Mouse | 265 | 80 | 9 | 52 | 0 |
| No. 4 | 85 | 46 | 8 | 47 | 0 |
| | 28 | 42 | 7 | 21 | 0 |
| | 9 | 33 | 3 | 9 | 0 |
| | 3 | 17 | 2 | 2 | 0 |
| | 1 | 5 | 2 | 1 | 0 |
| Mouse | 230 | 2 | 0 | 1 | 0 |
| No. 5 | 77 | 4 | 0 | 3 | 0 |
| | 25 | 2 | 0 | 0 | 0 |
| | 8 | 2 | 0 | 0 | 0 |
| | 3 | 1 | 1 | 0 | 0 |
| | 1 | 1 | 2 | 0 | 0 |
| Mouse | 210 | 13 | 2 | 11 | 0 |
| No. 6 | 70 | 10 | 0 | 7 | 0 |
| | 23 | 6 | 0 | 2 | 1 |
| | 3 | 1 | 0 | 0 | 0 |
| | 3 | 2 | 0 | 0 | 0 |
| | 1 | 2 | 0 | 0 | 0 |
| Intranasal | | | | | |
| Mouse | 430 | 58 | 0 | 52 | 6 |
| No. 1 | 160 | 52 | 0 | 58 | 1 |
| | 53 | 84 | 4 | 65 | 0 |
| | 18 | 84 | 2 | 52 | 0 |
| | 6 | 51 | 2 | 33 | 0 |
| | 2 | 52 | 0 | 20 | 0 |
| Mouse | 205 | 4 | 0 | 0 | 2 |
| No. 2 | 69 | 4 | 0 | 0 | 0 |
| | 83 | 1 | 0 | 0 | 0 |
| | 8 | 5 | 0 | 0 | 0 |
| | 2 | 3 | 0 | 0 | 0 |
| | 0.8 | 2 | 0 | 0 | 0 |
| Mouse No. 3 | 250 | 1 | 0 | 0 | 0 |
| | 83 | 0 | 0 | 0 | 0 |
| | 28 | 1 | 0 | 0 | 0 |
| | 9 | 1 | 0 | 0 | 0 |
| | 3 | 1 | 0 | 0 | 0 |
| | 1 | 1 | 0 | 0 | 0 |
| Mouse No. 4 | 280 | 2 | 0 | 1 | 0 |
| | 87 | 8 | 1 | 0 | 0 |
| | 29 | 1 | 0 | 0 | 0 |
| | 10 | 2 | 0 | 1 | 0 |
| | 3 | 0 | 0 | 0 | 0 |
| | 1 | 1 | 1 | 0 | 0 |
| Mouse No. 5 | 240 | 3 | 0 | 0 | 2 |
| | 80 | 1 | 0 | 0 | 0 |
| | 27 | 1 | 0 | 0 | 1 |
| | 9 | 1 | 1 | 0 | 0 |
| | 3 | 1 | 0 | 0 | 0 |
| | 1 | 1 | 1 | 1 | 0 |
| Mouse No. 6 | 125 | 0 | 0 | 0 | 0 |
| | 42 | 0 | 0 | 0 | 0 |
| | 14 | 0 | 0 | 0 | 0 |
| | 5 | 1 | 0 | 0 | 0 |
| | 2 | 0 | 0 | 0 | 1 |
| | 0.6 | 0 | 0 | 0 | 0 |

TABLE 4b
2°: Adβgal injected mice

| | | | | P511+ | P1.204+ |
|---|---|---|---|---|---|
| | Effector/ | | | cold L1210 | cold L1210 |
| | target | P511 | P1.204 | 50><1 ratio | 50><1 ratio |
| Subcutaneous | | | | | |
| Mouse No. 1 | 300 | 0 | 0 | 0 | 0 |
| | 100 | 0 | 0 | 0 | 0 |
| | 33 | 0 | 0 | 0 | 0 |
| | 11 | 0 | 0 | 0 | 0 |
| | 4 | 0 | 0 | 0 | 1 |
| | 1 | 0 | 0 | 0 | 0 |
| Mouse No. 2 | 230 | 4 | 3 | 0 | 1 |
| | 77 | 1 | 1 | 0 | 0 |
| | 25 | 1 | 4 | 0 | 0 |
| | 8 | 0 | 0 | 0 | 0 |
| | 3 | 0 | 0 | 0 | 0 |
| | 1 | 0 | 0 | 0 | 0 |
| Intra- | | | | | |
| peritoneal | | | | | |
| Mouse No. 1 | 220 | 1 | 2 | 1 | 0 |
| | 73 | 0 | 0 | 0 | 1 |
| | 24 | 1 | 1 | 1 | 2 |
| | 8 | 0 | 0 | 0 | 0 |
| | 3 | 0 | 0 | 0 | 1 |
| | 1 | 0 | 0 | 0 | 0 |
| Mouse No. 2 | 410 | 9 | 1 | 0 | 3 |
| | 137 | 1 | 1 | 0 | 2 |
| | 45 | 0 | 0 | 0 | 0 |
| | 15 | 0 | 1 | 0 | 0 |
| | 5 | 0 | 1 | 0 | 0 |
| | 2 | 0 | 0 | 0 | 0 |
| Intra- | | | | | |
| tracheal | | | | | |
| Mouse No. 1 | 310 | 0 | 0 | 0 | 0 |
| | 103 | 0 | 0 | 0 | 0 |
| | 34 | 0 | 0 | 0 | 0 |
| | 11 | 0 | 0 | 0 | 0 |
| | 4 | 0 | 0 | 0 | 0 |
| | 1 | 0 | 0 | 0 | 0 |
| Mouse No. 2 | 270 | 2 | 1 | 0 | 1 |
| | 90 | 1 | 0 | 0 | 0 |
| | 30 | 0 | 0 | 0 | 0 |
| | 10 | 0 | 0 | 0 | 0 |
| | 3 | 1 | 0 | 0 | 0 |
| | 1 | 1 | 0 | 0 | 0 |
| Intranasal | | | | | |
| Mouse | 400 | 0 | 0 | 0 | 0 |
| No. 1 | 18 | 1 | 0 | 0 | 1 |
| | 5 | 0 | 0 | 0 | 0 |
| | 2 | 0 | 0 | 0 | 0 |
| Mouse | 230 | 3 | 0 | 0 | 1 |
| No.2 | 77 | 2 | 0 | 0 | 0 |
| | 25 | 0 | 0 | 0 | 0 |
| | 9 | 0 | 0 | 0 | 0 |
| | 3 | 1 | 0 | 0 | 0 |
| | 1 | 0 | 0 | 0 | 0 |

| | | | | |
|---|---|---|---|---|
| spontaneous | 106 | 127 | 122 | 110 |
| maximum | 1041 | 971 | 957 | 988 |
| spont/max | 10% | 13% | 13% | 12% |

1. Defective recombinant adenovirus containing, inserted into its genome, a nucleic acid coding for a tumour-specific protein or peptide capable of inducing an immune protection and a destruction of the corresponding tumour cells by the immune system.

2. Defective recombinant adenovirus according to claim 1, characterized in that it contains a nucleic acid coding for a protein or peptide specific to a human tumour.

3. Defective recombinant adenovirus according to claim 1 or 2, characterized in that the nucleic acid inserted into its genome codes for all or part of an antigen specific to a melanoma.

4. Adenovirus according to claim 3, characterized in that the nucleic acid in question codes for a fragment of an antigen specific to a human melanoma comprising the portion presented to the CTL in combination with MHC-I molecules.

5. Adenovirus according to one of the preceding claims, characterized in that the nucleic acid codes for a protein, or a peptide derived therefrom, selected from the proteins Mage-1, Mage-3, Bage, Rage and Gage.

6. Defective recombinant adenovirus comprising, inserted into its genome, a nucleic acid coding for a peptide of the protein Mage-1 or Mage-3 comprising the portion presented to the CTL.

7. Defective recombinant adenovirus comprising, inserted into its genome, the sequence SEQ ID No. 1.

8. Defective recombinant adenovirus comprising, inserted into its genome, the sequence lying between residues 55 and 82 of the sequence SEQ ID No. 1.

9. Defective recombinant adenovirus comprising, inserted into its genome, the sequence SEQ ID No. 2.

10. Adenovirus according to one of the preceding claims, characterized in that it is chosen from the human serotypes Ad2 and Ad5.

11. Adenovirus according to one of claims 1 to 9, characterized in that it is chosen from canine serotypes.

12. Adenovirus according to one of the preceding claims, characterized in that it contains a deletion in the E1 region.

13. Adenovirus according to claim 11, characterized in that it contains, in addition, a deletion in the E4 region.

14. Adenovirus according to one of the preceding claims, characterized in that the nucleic acid is inserted into the E1 or E3 or E4 region.

15. Pharmaceutical composition comprising at least one adenovirus according to one of the preceding claims.

16. Use of an adenovirus according to one of claims 1 to 14, for the in vitro or ex vivo production of cytotoxic lymphocytes specific for human tumours.

17. Composition comprising cells infected with a defective recombinant adenovirus according to one of claims 1 to 14.

18. Composition according to claim 17, characterized in that it comprises antigen presenting cells (APC) infected with a defective recombinant adenovirus according to one of claims 1 to 14.

19. Method of preparing cytotoxic T cells specific for a tumour antigen comprising bringing a CTL cell precursor into contact with a population of cells infected with a virus according to one of claims 1 to 14.


SEQUENCE LISTING (3 sequences)

SEQ ID NO: 1 - DNA, 126 bp, Homo sapiens
ttgaattcgccgccatggagtccttgcagctggtctttggcattgacgtgaaggaagcagaccccaccggccactcctatgtccttgtcacctgcctaggtctctcctatgatggctagaattctt

SEQ ID NO: 2 - DNA, 44 bp, Homo sapiens
aattcgccgccatggaagtggaccccatcggccacttgtactag

SEQ ID NO: 3 - DNA, 44 bp, Homo sapiens
aattcgccgccatgctgccttatctagggtggctggtcttctag